(19) United States
(12) Patent Application Publication
(10) Pub. No.: US 2026/0092264 A1
(43) Pub. Date: Apr. 2, 2026

(54) NOVEL PROCESS FOR PURIFYING HEPARAN-N-SULFATASE

(71) Applicant: GREEN CROSS CORPORATION, Gyeonggi-do (KR)

(72) Inventors: Kwanyoung KO, Gyeonggi-do (KR); Dasom KIM, Gyeonggi-do (KR); Sulgi YOO, Gyeonggi-do (KR); Geunhye YEO, Gyeonggi-do (KR); Minjung KIM, Gyeonggi-do (KR); Dong Kyu JIN, Seoul (KR)

(21) Appl. No.: 19/110,710

(22) PCT Filed: Sep. 18, 2023
(86) PCT No.: PCT/KR2023/014028

§ 371 Date: Mar. 11, 2025

(30) Foreign Application Priority Data
Sep. 19, 2022 (KR) 10-2022-0117854

(51) Int. Cl. C12N 9/14; C07K 1/16; C07K 1/30; C07K 1/34

(52) U.S. Cl. CPC C12N 9/14; C07K 1/165; C07K 1/30; C07K 1/34; C12Y 310/01001

(57) ABSTRACT

The present invention is related to a method for purifying heparan-N-sulfatase from a heparan-N-sulfatase-containing solution including at least one impurity, the method comprising performing multi-mode chromatography (MMC) to obtain an eluate; and performing caprylate precipitation to obtain a supernatant.

The method according to the present invention is capable of very efficiently removing HCP (host cell proteins) and of greatly improving the purity and stability of purified heparan-N-sulfatase.

### TECHNICAL FIELD

The present invention relates to a novel method for purifying heparan-N-sulfatase (HNS) capable of greatly reducing the content of impurities and greatly improving the stability of heparan-N-sulfatase, a pharmaceutical composition comprising heparan-N-sulfatase prepared by the method, and a method for treating Sanfilippo syndrome using the same.

### BACKGROUND ART

Mucopolysaccharidoses (MPS) are rare hereditary lysosomal storage diseases caused by lack or deficiency of certain lysosomal enzymes.

Normal people with normal expression of lysosomal enzymes convert polysaccharide molecules into substances usable in vivo through a metabolic process by lysosomal enzymes. However, patients with lack or deficiency of lysosomal enzymes suffer from various types of diseases due to accumulation of polysaccharide molecules in cells, tissues, and organelles called lysosomes.

Sanfilippo syndrome, named after the American physician “Sanfilippo”, who first discovered the disease in 1963, is a type of mucopolysaccharidosis. Sanfilippo syndrome is an autosomal recessive genetic disease known as MPS III, and is characterized by clinically having no corneal opacity, and having mild physical changes such as hepatosplenomegaly or skeletal system changes, but very severe and progressive central nervous system symptoms.

Sanfilippo syndrome is caused by the deficiency of four different enzymes required to degrade polysaccharides, especially glycosaminoglycan (GAG) and is divided into MPS IIIA (Sanfilippo A), MPS IIIB (Sanfilippo B), MPS IIIC (Sanfilippo C) and MPS IIID (Sanfilippo D) depending on the deficient enzymes. The deficient enzymes and genetic map locus of each Sanfilippo syndrome are as follows.

Type A (MPS IIIA): heparan N-sulfatase-chromosome 17 (17q25.3)

Type B (MPS IIIB): N-acetyl-α-D-glucosaminidase-chromosome 17 (17q21)

Type C (MPS IIIC): acetyl-CoA:α-glucosaminide-N-acetyltransferase-chromosome-14

Type D (MPS IIID): N-acetyl-α-D-glucosaminide-6-sulfatase-chromosome 12 (12q14)

As can be seen from the above, MPS IIIA is caused by a deficiency of heparan-N-sulfatase, which is an enzyme involved in the degradation of heparan sulfate and hydrolyzes the sulfate moiety attached to the amino group of the glucosamine residue. Symptoms of MPS IIIA (Sanfilippo A) usually appear between the ages of 2 and 6, but may be diagnosed after the age of 13. In general, patients with MPS IIIA are known to have significant developmental delay and difficulty in long-term survival.

Currently, there is no approved therapy for MPS IIIA, but only symptomatic therapy for symptomatic relief is being conducted. Enzyme replacement therapy (ERT), that is, a therapy of administering externally prepared heparan-N-sulfatase to MPS IIIA patients is expected to be very useful for the treatment of MPS IIIA.

In order to treat MPS IIIA patients by enzyme replacement therapy using heparan-N-sulfatase, mass production of heparan-N-sulfatase is essential and processes of culturing and separating/purifying recombinant cells is required for mass production.

Separation and purification processes including immobilized metal affinity chromatography (IMAC), also known as metal chelate affinity chromatography (MCAC), cation exchange chromatography (CEX) and anion exchange chromatography (AEX) have been developed for the separation and purification of recombinantly produced heparan-N-sulfatase (see Korean Patent No. 2,286,260).

In addition, the separation and purification processes including anion exchange chromatography (AEX), hydrophobic interaction chromatography (HIC), hydroxyapatite chromatography (HA) and cation exchange chromatography (CEX) have been developed (see US Patent Publication No. 2012/0329133).

However, conventional processes still have problems of low heparan-N-sulfatase stability during the purification process, and deterioration in the quality and safety of the finally produced heparan-N-sulfatase due to insufficient removal of HCP (host cell proteins) and other impurities formed during production of heparan-N-sulfatase using recombinant cells. There is still an urgent need for a novel heparan-N-sulfatase separation/purification process that is capable of solving these problems.

### SUMMARY OF THE INVENTION

In order to solve the problems of the conventional process of separating and purifying heparan-N-sulfatase, it is one object of the present invention to provide a novel method and process for purifying heparan-N-sulfatase capable of minimizing deterioration in stability of heparan-N-sulfatase and greatly removing HCP and other impurities during the separation and purification process, a pharmaceutical composition comprising heparan-N-sulfatase prepared by the method, and a method for treating Sanfilippo syndrome using the same.

In order to accomplish the above object, the present invention provides a method for purifying heparan-N-sulfatase from a heparan-N-sulfatase-containing solution including at least one impurity, the method comprising performing multi-mode chromatography (MMC) to obtain an eluate; and performing caprylate precipitation to obtain a supernatant.

The heparan-N-sulfatase is a lysosomal enzyme known in the art called N-sulphoglucosamine sulphohydrolase (SGSH; EC 3.10.1.1); N-sulfoglucosamine sulfohydrolase; 2-desoxy-D-glucoside-2-sulfamate sulphohydrolase (sulphamate sulphohydrolase); heparin sulfamidase; sulfoglucosamine sulfamidase; sulfamidase; and HNS, rhHNS, sulfamidase, rhNS, and rhSGSH, and is particularly preferably derived from humans.

In the present invention, the human heparan-N-sulfatase has an amino acid sequence of SEQ ID NO: 1, or an amino acid sequence in which one or more amino acid residues are deleted at the N-terminus and/or C-terminus in the amino acid sequence of SEQ ID NO: 1, while having the activity of heparan-N-sulfatase, and in particular, is interpreted to have a sequence having a sequence homology of 90% or more, preferably 95% or more, more preferably 99% or more to the amino acid sequence of SEQ ID NO: 1, or the amino acid sequence in which one or more amino acid residues are deleted at the N-terminus and/or C-terminus in the amino acid sequence of SEQ ID NO: 1.

Amino acid sequence of human heparan-N-sulfatase
(SEQ ID NO: 1)

MSCPVPACCA LLLVLGLCRA RPRNALLLLA DDGGFESGAY
NNSAIATPHL DALARRSLLF RNAFTSVSSC SPSRASLLTG
LPQHQNGMYG LHQDVHHFNS FDKVRSLPLL LSQAGVRIGI
IGKKHVGPET VYPFDFAYTE ENGSVLQVGR NITRIKLLVR
KFLQTQDDRP FFLYVAFHDP HRCGHSQPQY GTFCEKFGNG
ESGMGRIPDW TPQAYDPLDV LVPYFVPNTP AARADLAAQY
TTVGRMDQGV GLVLQELRDA GVLNDTLVIF TSDNGIPFPS
GRINLYWPGT AEPLLVSSPE HPKRWGQVSE AYVSLLDLIP
TILDWFSIPY PSYAIFGSKT IHLTGRSLLP ALEAEPLWAT
VFGSQSHHEV TMSYPMRSVQ HRHFRLVHNL NFKMPFPIDQ
DFYVSPTFQD LLNRTTAGQP TGWYKDLRHY YYRARWELYD
RSRDPHETQN LATDPRFAQL LEMLRDQLAK WQWETHDPWV
CAPDGVLEEK LSPQCQPLHN EL

The heparan-N-sulfatase-comprising solution including one or more impurities is preferably, but is not limited to, a cell culture solution, wherein the cell culture solution is a culture solution of host cells capable of recombinantly producing heparan-N-sulfatase.

The host cells may be any host cells known in the art, and examples of the host cells include, but are not limited to, strains of the genus Bacillus, such as Escherichia coli, Bacillus subtilis and Bacillus thuringiensis, prokaryotic host cells such as Streptomyces, Pseudomonas (e.g. Pseudomonas putida), Proteus mirabilis or Staphylococcus (e.g. Staphylococcus carnosus), fungi such as Aspergillus species, yeasts such as Pichia pastoris, Saccharomyces cerevisiae, and Schizosaccharomyces and Neurospora crassa, other lower eukaryotic cells, higher eukaryote cells such as insect-derived cells, and cells derived from plants or mammals.

Preferably, the host cells may be monkey kidney cells (COS7), NSO cells, SP2/0, Chinese hamster ovary (CHO) cells, W138, baby hamster kidney (BHK) cells, MDCK, myeloma cell line cells, HuT 78 cells or 293 cells.

In addition, the cell culture solution may be clarified by removing cells and cell debris through depth filtration or the like.

In the present invention, the multi-mode chromatography may be implemented by simultaneously performing cation exchange chromatography (CEX) and hydrophobic interaction chromatography (HIC), but is not limited thereto.

Various impurities can be removed through multi-mode chromatography. In particular, selective elution of enzymes with different contents of M6P (mannose-6-phosphate) is possible, and the purity of the final purified heparan-N-sulfatase can be improved and the stability thereof can be remarkably increased by removing impurities with properties similar to those of heparan-N-sulfatase.

In the present invention, the resin for the multi-mode chromatography can be used without limitation as long as it is applicable to cation exchange chromatography and hydrophobic interaction chromatography simultaneously. For example, Capto MMC and Capto adhere resins having a structure of Formula 1 and Formula 2 may be used, but are not limited thereto.

| Capto MMC resin of Formula 1 | Capto adhere resin of Formula 2 |
|---|---|
| 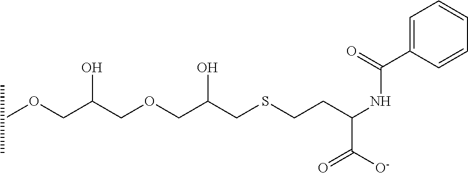 | 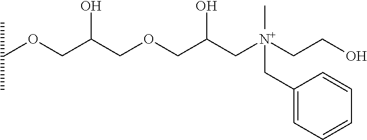 |

In performing multi-mode chromatography according to the present invention, the pH of the buffer (solution) for loading and equilibration is 4.5±1.0, preferably 4.5±0.5, and more preferably 4.5±0.1, but is not limited thereto.

The buffer contains 200±100 mM, preferably 200±50 mM, more preferably 200±20 mM NaCl, and is 5 to 100 mM, preferably 10 to 50 mM, more preferably 15 to 30 mM conventional acetate, phosphate, citrate, histidine, glycine, or Tris buffer, or the like without limitation, but is not limited thereto.

In the step of performing the multi-mode chromatography according to the present invention, the buffer for washing and elution after the resin binds to heparan-N-sulfatase is 5 to 100 mM, preferably 10 mM to 50 mM, more preferably 15 to 30 mM conventional histidine, glycine, acetate, phosphate, citrate, or Tris buffer, or the like, but is not limited thereto.

The pH of the buffer used in the washing step may be 5.0 to 8.0, preferably 6.0 to 7.5, more preferably 6.3 to 7.0.

In particular, the washing according to the present invention may comprise two or more washing steps. In this case, the pH of the buffer used in the subsequent washing step may be higher than the buffer used in the primary washing step.

Specifically, when the washing comprises two washing steps, the pH of the first washing step is 6.0 to 6.6, preferably 6.2 to 6.6, more preferably 6.4 to 6.5, and the pH of the second washing step is 6.6 to 7.2, preferably 6.7 to 7.0, more preferably 6.75 to 6.9, but is not limited thereto.

In addition, the pH of the buffer used in the elution step may be 6.6 to 9.0, preferably 6.9 to 8.8, more preferably 8.0 to 8.4, but is not limited thereto.

The step of performing caprylate precipitation to obtain a supernatant according to the present invention has the effect of increasing an impurity removal efficiency by efficiently removing HCP having a relatively low pH.

The caprylate precipitation is performed by adding caprylic acid or a salt thereof, preferably sodium caprylate, to a target solution, in a specific example, to a multi-mode chromatography eluate or affinity chromatography eluate to obtain a concentration of 1 to 50 mM, preferably 5 to 30 mM, more preferably 8 to 12 mM, at a pH of 3.5 to 6.0, preferably 4.0 to 5.5, more preferably 4.3 to 5.0.

The method for purifying heparan-N-sulfatase according to the present invention, between the performing multi-mode chromatography to obtain an eluate and the performing caprylate precipitation to obtain a supernatant, further comprises performing affinity chromatography to obtain an eluate.

Therefore, the method may comprise performing multi-mode chromatography to obtain an eluate; performing affinity chromatography to obtain an eluate; and performing caprylate precipitation to obtain a supernatant.

The step of using affinity chromatography according to the present invention comprises removing impurities, in particular, HCP such as Cathepsin X, having similar physical and chemical properties such as pI (isoelectric point) or hydrophobicity to heparan-N-sulfatase, to enhance the effect of removing impurities.

The resin used for affinity chromatography according to the present invention is preferably heparin sepharose or blue sepharose, but is not limited thereto.

The pH of the buffer (solution) used for loading, equilibration, washing and elution may be 4.5±1.0, preferably 4.5±0.5, more preferably 4.5±0.1, but is not limited thereto, and the buffer is 5 to 100 mM, preferably 10 to 50 mM, more preferably 15 to 30 mM conventional acetate, phosphate, citrate, histidine, glycine or Tris buffer, or the like, without limitation, but is not limited thereto.

In particular, the buffer used for washing may contain 150±100 mM, preferably 150±50 mM, more preferably 150±20 mM NaCl, and the buffer used for elution may contain 300±100 mM, preferably 300±50 mM, more preferably 300±20 mM NaCl.

The method for purifying heparan-N-sulfatase according to the present invention, prior to performing multi-mode chromatography to obtain an eluate, may further comprise performing primary anion exchange chromatography (AEX) to obtain an eluate, solvent/detergent treatment, and performing cation exchange chromatography (CEX) to obtain an eluate.

Therefore, the method may comprise performing primary anion exchange chromatography (AEX) to obtain an eluate; solvent/detergent treatment; performing cation exchange chromatography (CEX) to obtain an eluate; performing multi-mode chromatography to obtain an eluate; performing affinity chromatography to obtain an eluate; and performing caprylate precipitation to obtain a supernatant.

The primary anion exchange chromatography is a step for maximally recovering heparan-N-sulfatase from the cell culture solution, and the resin used for the primary anion exchange chromatography according to the present invention is either a weak anion exchange resin or a strong anion exchange resin, and examples of the resin include, but are not limited to, weak anion resins such as DEAE Sepharose, and strong anion resins such as Q Sepharose, Fractogel TMAE (M), Fractogel TMAE (S) and Poros XQ, and the like.

The pH of the buffer (solution) for loading, equilibration, washing and elution during the primary anion exchange chromatography according to the present invention is 7.5±1.0, preferably 7.5±0.7, more preferably 7.5±0.5, but is not limited thereto, and the resin is 5 to 100 mM, preferably 10 to 70 mM, more preferably 40 to 60 mM conventional Tris, acetate, phosphate, citrate, histidine or glycine buffer, or the like without limitation, but is not limited thereto. Also, the elution buffer may contain 200±100 mM, preferably 200±70 mM, and more preferably 200±50 mM NaCl.

The solvent/detergent treatment is a step for inactivating virus preferably using a combination of polysorbate, specifically polysorbate 20 and/or 80, and tri-n-butyl-phosphate (TnBP), but is not limited thereto.

The solvent/detergent treatment is performed at a pH of 7.5±1.0, preferably 7.5±0.5, more preferably 7.5±0.2, at 18 to 28° C., preferably 20 to 25° C., for 1 hour or more and 6 hours or less.

The cation exchange chromatography is a process for maximally recovering heparan-N-sulfatase and removing impurities such as process-related impurities and HCP formed during the solvent/detergent treatment.

The resin used in the cation exchange chromatography according to the present invention may be either a weak cation exchange resin or a strong cation exchange resin, examples thereof include carboxymethyl (CM), sulfopropyl (SP) and methyl sulfonate (S) resins, and the resin is preferably SP Sepharose or the like, but is not limited thereto.

The pH of the buffer (solution) for loading, equilibration, washing and elution during the cation exchange chromatography according to the present invention is 4.5±0.7, preferably 4.5±0.5, more preferably 4.5±0.1, but is not limited thereto, and the resin is 5 to 100 mM, preferably 10 to 50 mM, more preferably 15 to 30 mM conventional acetate, phosphate, citrate, histidine, glycine or Tris buffer, or the like without limitation, but is not limited thereto.

In addition, the buffer for loading or equilibration may contain 100±70 mM, preferably 100±50 mM, more preferably 100±20 mM NaCl, and the elution buffer may contain 200±100 mM, preferably 200±50 mM NaCl, more preferably 200±20 mM NaCl.

The method for purifying heparan-N-sulfatase according to the present invention, after performing caprylate precipitation to obtain a supernatant, may further comprise performing secondary anion exchange chromatography (AEX) to obtain an eluate.

Therefore, the method may comprise performing primary anion exchange chromatography (AEX) to obtain an eluate; solvent/detergent treatment; performing cation exchange chromatography (CEX) to obtain an eluate; performing multi-mode chromatography to obtain an eluate; performing affinity chromatography to obtain an eluate; performing caprylate precipitation to obtain a supernatant; and performing secondary anion exchange chromatography (AEX) to obtain an eluate.

The secondary anion exchange chromatography is a process for maximally recovering heparan-N-sulfatase and removing process-related impurities such as heparin, caprylate or solvent/detergent. The resin used in the anion exchange chromatography according to the present invention is preferably a strong anion exchange resin such as Q Sepharose, Fractogel TMAE (M), Fractogel TMAE (S) or Poros XQ, but is not limited thereto.

The pH of the buffer (solution) for loading, equilibration, washing and elution during the secondary anion exchange chromatography according to the present invention is 7.5±1.0, preferably 7.5±0.7, more preferably 7.5±0.5, but is not limited thereto, and the resin is 5 to 100 mM, preferably 10 to 50 mM, more preferably 15 to 30 mM conventional histidine, glycine, acetate, phosphate, citrate or Tris buffer or the like, without limitation, but is not limited thereto.

In addition, the equilibration buffer may contain 50±20 mM, preferably 50±10 mM, more preferably 50±5 mM NaCl, and the elution buffer may contain 150±50 mM, preferably 150±30 mM, more preferably 150±10 mM NaCl.

The method for purifying heparan-N-sulfatase according to the present invention, after performing secondary anion exchange chromatography (AEX) to obtain an eluate, may further comprise nanofiltration.

Therefore, the method may comprise performing primary anion exchange chromatography (AEX) to obtain an eluate; solvent/detergent treatment; performing cation exchange chromatography (CEX) to obtain an eluate; performing multi-mode chromatography to obtain an eluate; performing affinity chromatography to obtain an eluate; performing caprylate precipitation to obtain a supernatant; performing secondary anion exchange chromatography (AEX) to obtain an eluate; and nanofiltration.

The nanofiltration aims at removing viruses and may be performed using a nanofilter commonly used to remove virus.

The method for purifying heparan-N-sulfatase according to the present invention may further comprise ultrafiltration/diafiltration (UF/DF); at least one time selected from the steps consisting of before performing primary anion exchange chromatography (AEX) to obtain an eluate; between performing caprylate precipitation to obtain a supernatant and performing secondary anion exchange chromatography (AEX) to obtain an eluate; and after nanofiltration.

Therefore, when ultrafiltration/diafiltration (UF/DF) is comprised in all the steps which are before performing primary anion exchange chromatography (AEX) to obtain an eluate, between performing caprylate precipitation to obtain a supernatant and performing secondary anion exchange chromatography (AEX) to obtain an eluate, and after nanofiltration, the overall method may comprise: primary ultrafiltration/diafiltration (UF/DF);
performing primary anion exchange chromatography (AEX) to obtain an eluate;
solvent/detergent treatment;
performing cation exchange chromatography (CEX) to obtain an eluate;
performing multi-mode chromatography to obtain an eluate;
performing affinity chromatography to obtain an eluate;
performing caprylate precipitation to obtain a supernatant;
secondary ultrafiltration/diafiltration (UF/DF);
performing secondary anion exchange chromatography (AEX) to obtain an eluate;
nanofiltration; and tertiary ultrafiltration/diafiltration (UF/DF).

The primary ultrafiltration/diafiltration aims at concentrating the cell culture solution, preferably the cell culture solution that has undergone a purification process, and exchanging the buffer to reduce the volume of the target culture solution, shorten the column loading time, and improve the convenience of the subsequent process.

The cut-off value of the membrane used in the primary ultrafiltration/diafiltration may be 10 to 100 kDa, preferably 20 to 70 kDa, more preferably 30 to 50 kDa, but is not limited thereto.

The pH of the buffer used in the primary ultrafiltration/diafiltration may be 7.5±1.0, preferably 7.5±0.7, more preferably 7.5±0.5, but is not limited thereto, and is used 5 to 100 mM, preferably 10 to 80 mM, more preferably 30 to 70 mM conventional Tris, histidine, glycine, acetate, phosphate or citrate buffer, or the like without limitation, but is not limited thereto.

The secondary ultrafiltration/diafiltration is performed between the caprylate precipitation and the secondary anion exchange chromatography and aims at converting pH and conductivity.

The cut-off value of the membrane used in the secondary ultrafiltration/diafiltration may be 10 to 100 kDa, preferably 20 to 70 kDa, and more preferably 30 to 50 kDa, but is not limited thereto.

The pH of the buffer used in the secondary ultrafiltration/diafiltration may be 7.5±1.0, preferably 7.5±0.7, more preferably 7.5±0.5, but is not limited thereto, and is used 5 to 50 mM, preferably 10 to 40 mM, more preferably 15 to 30 mM conventional histidine, glycine, acetate, phosphate, citrate or Tris buffer, or the like without limitation, but is not limited thereto.

When the conductivity of the secondary ultrafiltration/diafiltration is 10 mS/cm or less, preferably 8 mS/cm or less, and more preferably 6 mS/cm or less, the process solution is recovered and the pH is adjusted to 7.5±1.0.

The tertiary ultrafiltration/diafiltration is performed after nanofiltration and aims at finally concentrating the result to a high concentration and exchanging the buffer.

The cut-off value of the membrane used in the tertiary ultrafiltration/diafiltration may be 10 to 100 kDa, preferably 20 to 70 kDa, and more preferably 30 to 50 kDa, but is not limited thereto.

The pH of the buffer used in the tertiary ultrafiltration/diafiltration may be 8.0±1.0, preferably 8.0±0.7, more preferably 8.0±0.5, but is not limited thereto, and is used 1 to 20 mM, preferably 2 to 10 mM, more preferably 3 to 8 mM conventional histidine, glycine, acetate, phosphate, citrate or Tris buffer, or the like without limitation, but is not limited thereto.

Unless otherwise specified, all purification steps according to the present invention are performed at room temperature, specifically 15 to 30° C., preferably 18 to 25° C.

### BRIEF DESCRIPTION OF DRAWINGS

FIG. 1 is a chromatogram illustrating a process of purifying heparan-N-sulfatase using primary anion exchange chromatography.

FIG. 2 shows the result of SDS-PAGE of each step solution of the primary anion exchange chromatography.

FIG. 3 is a chromatogram illustrating a process of purifying heparan-N-sulfatase using cation exchange chromatography.

FIG. 4 shows the result of SDS-PAGE of each step solution of cation exchange chromatography.

FIG. 5 is a chromatogram depending on the pH of the eluate in multi-mode chromatography.

FIG. 6 is a diagram illustrating the result of SDS-PAGE depending on the pH of the eluate in multi-mode chromatography.

FIG. 7 shows the result of elution in affinity chromatography.

FIG. 8 shows an overall process for purifying heparan-N-sulfatase according to an embodiment of the present invention.

### DETAILED DESCRIPTION AND PREFERRED EMBODIMENTS OF THE INVENTION

Hereinafter, the present invention will be described in more detail with reference to examples. However, it will be obvious to those skilled in the art that these examples are provided only for illustration of the present invention and should not be construed as limiting the scope of the present invention.

Unless defined otherwise, all technical and scientific terms used herein have the same meanings as appreciated by those skilled in the field to which the present invention pertains.

### EXAMPLE

### Example 1: Clarification of Cell Culture Solution

The cell culture solution containing recombinantly produced heparan-N-sulfatase and one or more impurities was subjected to depth filtration using a depth filter.

Specifically, distilled water was thoroughly allowed to flow into a depth filter Cat. No. MDOHCO54H1 or Cat. No. MXOHCO27H1 from Merck KGaA and a depth filter Cat. No. NP6PDK516 or Cat. No. NP5LPDD16 from Pall Corporation to remove the solution contained in the filter, pH 7.5, 50 mM Tris buffer was allowed to flow to achieve an equilibrium state and then the cell culture solution was filtered in the range of 2.0 bar.

The recovery rate was 95% or more and the removal capacity of HCP was about 0.4 LRV (log reduction value).

### Example 2: Primary Ultrafiltration/Diafiltration (UF/DF)

The clarified cell culture solution was subjected to primary ultrafiltration/diafiltration to perform concentration and buffer exchange.

Specifically, pH 7.5, 50 mM Tris buffer was allowed to flow into a Pellicon 3 Ultracel C screen (Cat. No. P3C030C01) having a cut-off value of 30 to 50 kDa from Merck KGaA or Omega T-series 30 kDa (OS030T12) membrane from Poll Corporation, to achieve an equilibrium state, and the culture filtrate was concentrated 10 times compared to the initial volume, was concentrated up to 13 times compared to the initial volume through buffer exchange with 3 DV (diafiltration volume) or more of a pH 7.5, 50 mM Tris buffer and then recovered.

The concentration factor was 7 to 13 times and the buffer exchange volume was 3 DV or more. Finally, the recovery rate was 95% or more and the removal capacity of HCP was about 0.1 LRV (log reduction value) or more.

### Example 3: Primary Anion Exchange Chromatography

### 3.1 VALIDATION OF BASIC ANION EXCHANGE CHROMATOGRAPHY (AEX)

The primarily ultrafiltered/diafiltered solution was subjected to anion exchange chromatography using, as a strong anion exchange resin, Q Sepharose 6 Fast Flow, Fractogel EMD TMAE (M), or Poros XQ, and using, as a weak anion exchange resin, DEAE Sepharose Fast Flow.

Specifically, the resin was subjected to CIP (cleaning in place) with 5 CV (column volume) of 0.5N NaOH, 15 CV of pH 7.5±0.5, 50 mM Tris equilibration buffer (EQ buffer) was allowed to flow to achieve an equilibrium state, and the primary UF/DF solution was loaded. Then, pH 7.5±0.5, 50 mM Tris equilibration buffer was injected in 5 CV to achieve an equilibrium state again, 5 CV of 50 mM Tris elution buffer (pH 7.5±0.5, 200±50 mM NaCl) was injected and the eluate was collected up to 2.5 CV (132.5 mL) when the UV signal during the chromatography was 50 mAu.

Then, 5 CV of pH 7.5±0.5, 2,000±200 mM NaCl, and 50 mM Tris column wash buffer (CW buffer) were injected and subjected to CIP with 5 CV of 0.5 N NaOH, and 15 CV of equilibration buffer was allowed to flow to achieve an equilibrium state.

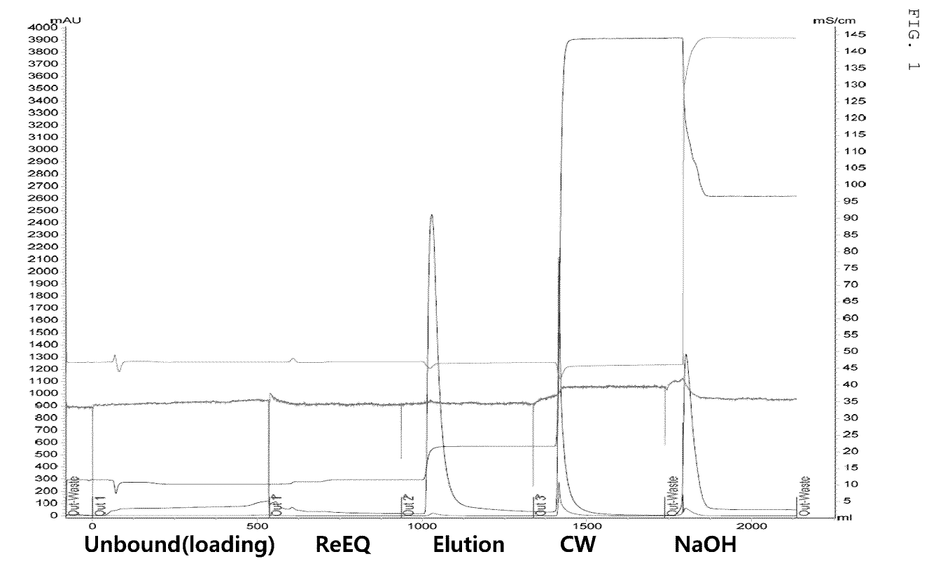

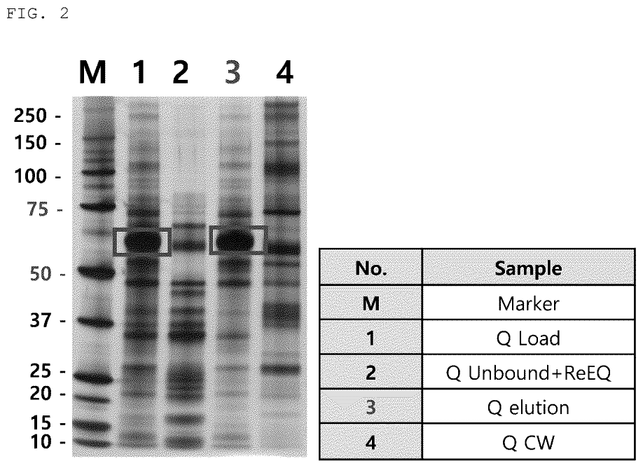

The result of anion exchange chromatography under the conditions described above showed that, when unbound, CW and NaOH were injected in addition to elution in the chromatogram, UV signals were detected, which means that impurities were removed (see FIG. 1), and the result of SDS-PAGE showed that, in the eluate (Lane 3), at least as much impurities as Lane 2 and Lane 4 were removed compared to the load sample (Lane 1) (see FIG. 2).

Overall, the yield was about 90% or more, the purity was about 70% or more, and the HCP removal capacity was about 0.2 LRV or more. All the resins used had similar recovery rates upon process optimization. Therefore, Q Sepharose is preferred in consideration of process robustness and economic feasibility.

### 3.2 Optimization of Anion Exchange Chromatography (AEX)

As shown in Table 1, the performance of the AEX process was tested while varying the process pH and NaCl concentration in the elution buffer.

Specifically, the test was conducted with a difference of ±0.5 based on the initial experimental pH (pH 7.5), and stepwise elution was performed with 100, 150, and 200 mM NaCl concentrations to determine the elution performance of the target protein, heparan-N-sulfatase depending on the NaCl concentration in the elution buffer at each pH.

TABLE 1
AEX performance depending on process pH
and NaCl concentration in elution buffer

| | Sample | | Target protein content (ELISA) | |
|---|---|---|---|---|
| Process | (NaCl | | Content | Yield |
| pH | concentration) | Volume | (mg/mL) | (%) |
| 7.0 | Load | 60.0 | 1.851 | N/A |
| | Unbound | 210.0 | 0.099 | 18.7 |
| | Elution 1 (100 mM) | 300.0 | 0.279 | 75.4 |
| | Elution 2 (150 mM) | 150.0 | 0.046 | 12.4 |
| | Elution 3 (200 mM) | 150.0 | 0.003 | 0.4 |
| | CW | 150.0 | 0.003 | 0.4 |
| 7.5 | Load | 60.0 | 1.822 | N/A |
| | Unbound | 210.0 | 0.084 | 16.2 |
| | Elution 1 (100 mM) | 300.0 | 0.269 | 73.9 |
| | Elution 2 (150 mM) | 300.0 | 0.065 | 17.9 |
| | Elution 3 (200 mM) | 150.0 | 0.006 | 0.9 |
| | CW | 150.0 | 0.003 | 0.4 |
| 8.0 | Load | 60.0 | 1.896 | N/A |
| | Unbound | 210.0 | 0.016 | 2.9 |
| | Elution 1 (100 mM) | 300.0 | 0.344 | 90.8 |
| | Elution 2 (150 mM) | 300.0 | 0.046 | 12.1 |
| | Elution 3 (200 mM) | 150.0 | 0.006 | 0.8 |
| | CW | 150.0 | 0.003 | 0.4 |

| | | |
|---|---|---|
| Acceptance Criteria | N/A | ≥80 |

The result showed that heparan-N-sulfatase bound to the resin at pH 7.0 to 8.0 and a large proportion of heparan-N-sulfatase was eluted from 100 mMv NaCl. Also, most of the heparan-N-sulfatase was recovered from the 150 mMv NaCl fraction. Therefore, it was found that satisfactory results could be obtained when the NaCl concentration in the elution buffer was 100 mMv, preferably 150 mMv NaCl or more.

### Example 4: Solvent/Detergent Treatment

Viruses were inactivated in the eluate obtained by the primary AEX through solvent/detergent treatment (S/D treatment).

Specifically, a S/D stock solution was added to the eluate obtained by the primary AEX to achieve 1% Polysorbate 80 and 0.3% TnBP, and stirred at 20 to 25° C. for 1 hour.

At this time, the S/D treatment was performed under three conditions of pH 7.3, 7.5 and 7.7 for up to 6 hours, and it was confirmed that sufficient S/D treatment was possible within the pH range defined above. However, when the treatment time exceeds 6 hours, eluate turbidity may cause a process risk. Therefore, it is preferable to adjust the treatment time to less than 6 hours.

### Example 5: Cation Exchange Chromatography

### 5.1 Validation of Basic Cation Exchange Chromatography (CEX)

Cation exchange chromatography was performed using SP Sepharose Fast Flow resin to maximize the recovery of heparan-N-sulfatase from the S/D-treated solution and to remove impurities, especially solvents and detergents, which are process impurities.

Specifically, the resin was subjected to CIP (cleaning in place) with 5 CV (column volume) of 0.5 N NaOH, 10 CV of 20 mM sodium acetate (S.A.) equilibration buffer (EQ buffer, pH 4.5±0.1, 100±20 mM NaCl) was allowed to flow to achieve an equilibrium state, and the S/D-treated solution was loaded.

Then, 5 CV of 20 mM sodium acetate equilibration buffer (pH 4.5±0.1, 100±20 mM NaCl) was injected to achieve an equilibrium state again, 5 CV of 20 mM sodium acetate elution buffer (pH 4.5±0.1, 200±20 mM NaCl) was injected, and the eluate was initially collected in an amount of 3 CV and was then discarded as waste in a remaining amount of 2 CV.

Then, 5 CV of 20 mM sodium acetate column wash buffer (CW buffer, pH 4.5±0.1, 2000±200 mM NaCl) was injected, CIP was performed with 5 CV of 0.5N NaOH, and 10 CV of equilibration buffer was allowed to flow to achieve an equilibrium state.

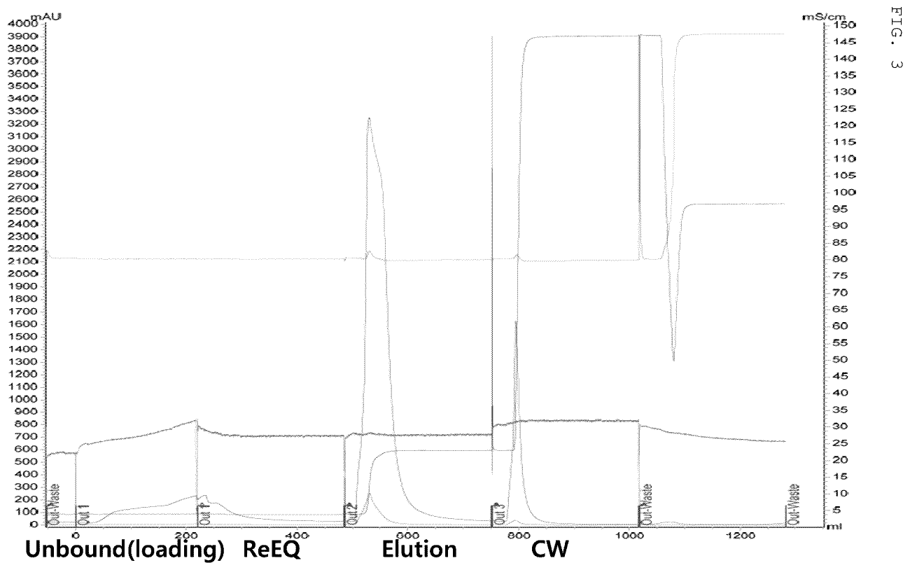

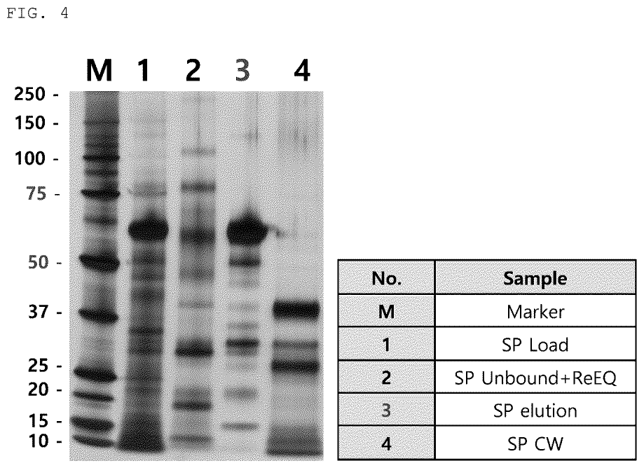

The result of cation exchange chromatography under the conditions described above showed that, when unbound, CW and NaOH were injected in addition to elution in the chromatogram, UV signals were detected, which means that impurities were removed (see FIG. 3), as can be seen from the result of SDS-PAGE. In the eluate (Lane 3), at least as much impurities as Lane 2 and Lane 4 were removed compared to the load sample (Lane 1) (see FIG. 4) and the purity increased by about 20% compared to the primary anion exchange chromatography eluate.

Overall, the yield was about 90% or more, the purity was about 93% or more, and the HCP removal capacity was about 1.2 LRV or more.

### 5.2 Optimization of Cation Exchange Chromatography (CEX)

As shown in Table 2, the performance of the CEX process was tested while varying the process pH and NaCl concentration in the elution buffer.

Specifically, the test was conducted within the range of 4.0 to 5.4, based on the initial experimental pH (pH 4.5), and a test was performed depending on the concentration of 2 to 300 mM, to determine the elution performance of the target protein, heparan-N-sulfatase, depending on the NaCl concentration in the elution buffer at each pH.

TABLE 2
CEX performance depending on process pH
and NaCl concentration in elution buffer

| Process | | pH | Condition |
|---|---|---|---|
| pH | Equilibrium | 4.5 | 20 mM S.A. + 100 mM NaCl (pH 4.5) |
| Study 1 | Loading | | 20 mM S.A. (pH 4.5) ≈ 10 mS/cm |
| | Re-equilibrium | | 20 mM S.A. + 100 mM NaCl (pH 4.5) |
| | Elution | | 20 mM S.A. + 200 mM NaCl (pH 4.5) |
| | Column Wash | | 20 mM S.A. + 2M NaCl (pH 4.5) |
| | Equilibrium | 5.0~5.4 | 20 mM S.A. + 20 mM NaCl (pH 5.0~5.4) |
| | Loading | | 20 mM S.A. (pH 5.0~5.4) ≈ 4 mS/cm |
| | Re-equilibrium | | 20 mM S.A. + 20 mM NaCl (pH 5.0~5.4) |
| | Elution | | 20 mM S.A. + 50~300 mM NaCl (pH 5.0~5.4) |
| | Column Wash | | 20 mM S.A. + 2M NaCl (pH 5.0~5.4) |
| pH | Equilibrium | 4.4~4.6 | 20 mM S.A. + 100 mM NaCl (pH 4.4~4.6) |
| study 2 | Loading | | 20 mM S.A. (pH 4.5) ≈ 10 mS/cm |
| | Re-equilibrium | | 20 mM S.A. + 100 mM NaCl (pH 4.4~4.6) |
| | Elution | | 20 mM S.A. + 200 mM NaCl (pH 4.4~4.6) |
| | Column Wash | | 20 mM S.A. + 2M NaCl (pH 4.4~4.6) |

As can be seen from Table 3, the result showed that the optimal NaCl concentration in the elution buffer at this time was 50 (at pH 5.4) to 210 mM NaCl (at pH 4.6 or less).

TABLE 3
Results of elution depending on process pH and
NaCl concentration in the elution buffer

| Process | Run No. | pH | Yield (%) | Purity (%) |
|---|---|---|---|---|
| pH study 1 | 3 | 4.5 | 86.7 | 89.7 |
| | 4 | 5.0 | 87.5 | 81.5 |
| | 4-1 | 5.0 | 64.6 | 91.3 |
| | 5 | 5.4 | 71.6 | 94.9 |
| pH study 2 | 1 | 4.4 | 88.5 | 99.8 |
| | 2 | 4.6 | 82.3 | 97.3 |

| | | |
|---|---|---|
| Acceptance criteria | ≥78.0 | ≥83.0 |

### Example 6: Multi-Mode Chromatography

Multi-mode chromatography was performed using Capto MMC resin capable of simultaneously performing cation exchange chromatography (CEX) and hydrophobic action chromatography (HIC) in order to more efficiently remove impurities from the CEX eluate and selectively purify heparan-N-sulfatase containing M6P (mannose 5-phosphate).

Specifically, the resin was subjected to CIP with 5 CV of 0.5 N NaOH, 10 CV of 20 mM sodium acetate (S.A.) equilibration buffer (EQ buffer, pH 4.5, 200 mM NaCl) was allowed to flow to achieve an equilibrium state, and the CEX eluate was loaded.

Then, 5 CV of 20 mM histidine equilibration buffer (pH 5.5±0.1) was injected to achieve a re-equilibrium state, and the column was primarily washed with 5 CV of 20 mM histidine primary wash buffer (pH 6.5±0.1) and then secondarily washed with 10 CV of 20 mM histidine secondary wash buffer (pH 6.85±0.1).

When the pH of the wash buffer was 6.5 to 6.9, the solution after washing contained a great amount of HCP, but the content of heparan-N-sulfatase was rather low. The washing was performed by two steps, the pH of the first wash buffer was 6.45 and the pH of the secondary wash buffer was increased to 6.85, to more efficiently remove HCP and increase the recovery rate of heparan-N-sulfatase.

Then, 10 CV of histidine elution buffer (pH 6.8±0.1 to 8.3±0.1, 20 mM) was injected and all of the eluates were recovered.

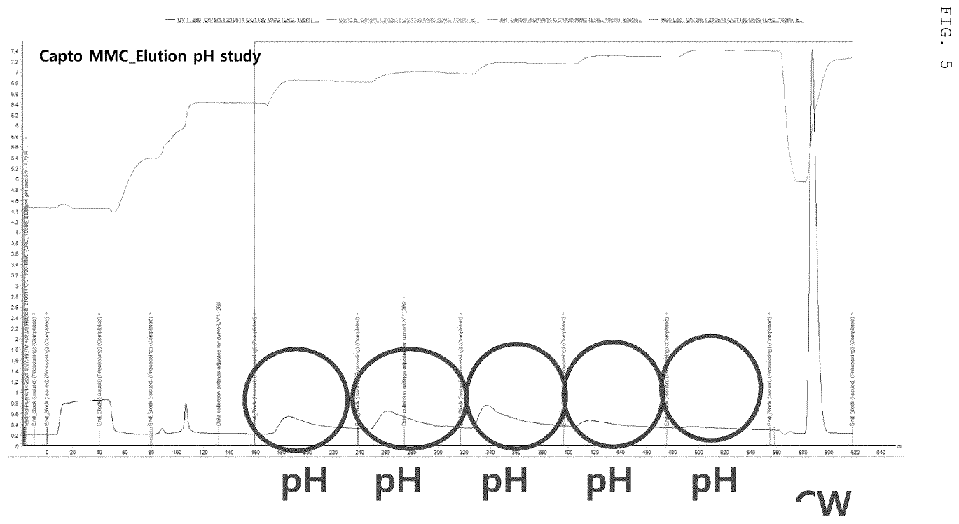

The result showed that the target protein, heparan-N-sulfatase was first recovered at 6.7 of an elution buffer pH, and proper recover of heparan-N-sulfatase was possible up to 8.4 of an elution buffer pH (See FIG. 5).

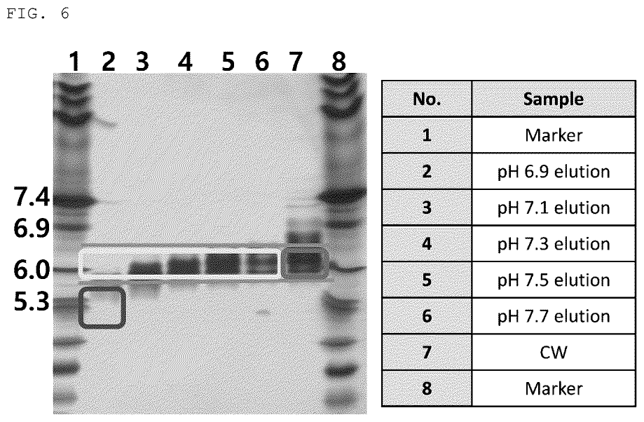

In addition, as the pH of the elution buffer increased, the range of pI of heparan-N-sulfatase gradually increased, and there was no significant difference in the FGly content between fractions, but the M6P content decreased as the elution pH increased. That is, heparan-N-sulfatase having a lower pI value contains a larger amount of M6P (see FIG. 6 and Table 4).

TABLE 4
Purification efficacy of M6P depending on pH of elution buffer

| | FGly | M6P | HCP | Purity |
|---|---|---|---|---|
| Sample pH | (%) | (mol/mol) | (ppm) | (%) |
| 6.9 | 54.24 | 3.43 | 1365 | 97.8 |
| 7.1 | 54.72 | 3.10 | 485 | 98.5 |
| 7.3 | 54.64 | 2.97 | 304 | 98.4 |
| 7.5 | 53.82 | 2.61 | 223 | 97.9 |
| 7.7 | 54.45 | 2.56 | 199 | 97.8 |
| CW | 57.85 | 1.93 | 729 | 97.4 |

Then, 5 CV of 20 mM histidine column wash buffer (CW buffer, pH 7.5, 2,000 mM NaCl) was injected, CIP was performed with 5 CV of 0.5 N NaOH, and 10 CV of equilibration buffer was allowed to flow to achieve an equilibrium state.

Overall, the yield was about 70-90% or more, the purity was about 97% or more, and the HCP removal capacity was about 1.8 LRV or more.

### Example 7: Affinity Chromatography

### 7.1 Validation of Basic Affinity Chromatography

Affinity chromatography was performed using heparin Sepharose or blue Sepharose resins to remove impurities, for example, Cathepsin X, which are most contained in the MMC eluate.

Specifically, the resin was subjected to CIP with 5 CV of 0.1 N NaOH, 10 CV of 20 mM sodium acetate (S.A.) equilibration buffer (EQ buffer, pH 4.5, 200 mM NaCl) was allowed to flow to achieve an equilibrium state, and the MMC eluent was loaded.

Subsequently, 5 CV of 20 mM sodium acetate (S.A.) equilibration buffer (pH 4.5±0.1) was injected to achieve a re-equilibrium state and the column was secondarily washed with 10 CV of 20 mM sodium acetate wash buffer (pH 4.5±0.1, 150±20 mM NaCl).

Then, 10 CV of 20 mM sodium acetate elution buffer (pH 4.5±0.1, 300±20 mM NaCl) was injected, all of the eluate was recovered, 5 CV of 20 mM sodium acetate wash buffer (CW buffer, pH 4.5±0.1, 2,000±200 mM NaCl) was injected, CIP was performed with 5 CV of 0.1 N NaOH, and 10 CV of equilibration buffer was allowed to flow to achieve an equilibrium state.

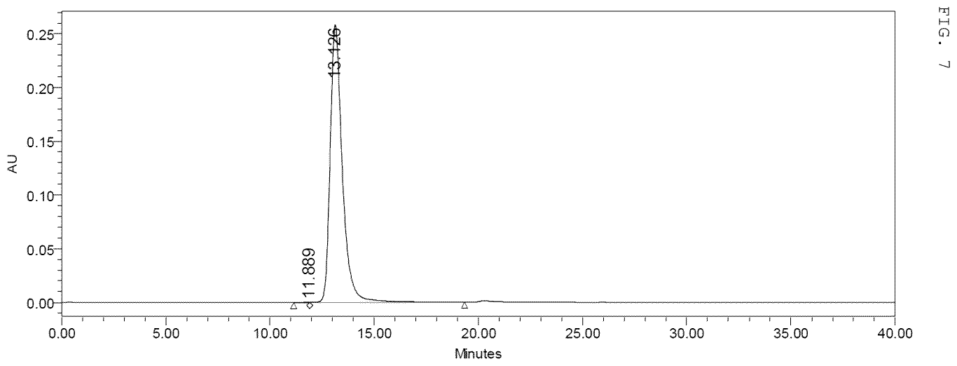

The result of affinity chromatography according to the above process showed that heparan-N-sulfatase was purified to a very high purity, as shown in FIG. 7.

Overall, the yield was about 90% or more, the purity was about 99% or more, and the HCP removal capacity was about 1.0 LRV or more.

### 7.2 Optimization of Affinity Chromatography

As shown in Table 5, the performance of the affinity chromatography was tested while varying the process pH and NaCl concentration in the elution buffer.

Specifically, based on the initial condition test, the pH was set to pH 4.5±0.1, the center NaCl concentration of the wash buffer was set to 150 mM, and the center NaCl concentration of the elution buffer was set to 300 mM.

TABLE 5
Results of elution depending on process pH and
NaCl concentration in the elution buffer

| | | HNS content | | | | |
|---|---|---|---|---|---|---|
| Run | | | Yield | HCP content | | Purity |
| No. | Sample | mg/mL | (%) | ng/mg | LRV | (%) |
| Run 1 | Load | 0.5 | — | 947.4 | — | 98.31 |
| | Wash (130 mM) | 0.01 | 1.8 | — | — | — |
| | Elution (300 mM) | 0.54 | 86.9 | 154.8 | 0.8 | 100 |
| Run 2 | Load | 0.50 | — | 947.4 | — | — |
| | Wash (150 mM) | 0.01 | 1.84 | — | — | — |
| | Elution (300 mM) | 0.55 | 88.2 | 158.0 | 0.8 | 100 |
| Run 3 | Load | 0.50 | | 947.4 | — | — |
| | Wash (170 mM) | 0.01 | 1.94 | — | — | — |
| | Elution (300 mM) | 0.55 | 89.0 | 115.2 | 0.9 | 100 |
| Run 4 | Load | 0.50 | — | 947.40 | — | — |
| | Wash (150 mM) | 0.01 | 1.94 | — | — | — |
| | Elution (280 mM) | 0.49 | 79.2 | 77.80 | 1.1 | 99.99 |
| Run 5 | Load | 0.50 | — | 947.4 | — | — |
| | Wash (150 mM) | 0.01 | 2.05 | — | — | — |
| | Elution (320 mM) | 0.58 | 93.1 | 66.5 | 1.2 | 99.97 |

| | | | | | |
|---|---|---|---|---|---|
| Acceptance criteria | N/A | ≥70 | N/A | ≥0.5 | ≥96 |

As can be seen from Table 5, the result showed that HCP was efficiently removed and heparan-N-sulfatase was obtained with high purity when the NaCl concentration of the wash buffer was in the range of 130 to 170 mM and the NaCl concentration in the elution buffer was in the range of 280 to 320 mM.

### Example 8: Caprylate Precipitation

Caprylate precipitation was performed to precipitate and remove HCP having a lower pI.

Specifically, a 500 mM stock solution of sodium caprylate was prepared and added to the affinity chromatography eluate to adjust a concentration to 10 mM.

Then, precipitation was allowed to occur under conditions of pH of 4.5±0.1 and 20 to 25° C. for 1 to 3 hours while stirring at a speed of 200±20 rpm, and the precipitated impurities were removed by filtration.

As can be seen from Table 6, the result showed that the content of HCP, which is most of the impurities, was greatly reduced when caprylate precipitation was performed compared to the case where the caprylate precipitation was not performed. Almost no Cathepsin X, the main HCP, was detected and lysosomal Pro-X carboxypeptidase was greatly reduced.

TABLE 6
Impurity removal effect by caprylate precipitation

Measured value (ppm)

| | Without | With |
|---|---|---|
| Impurity (HCP) | precipitation | precipitation |
| Cathepsin X | 3,100 | N.D |
| Lysosomal Pro-X carboxypeptidase | 2,758 | 705 |
| Alpha-mannosidase | 839 | 286 |
| Alpha-L-fucosidase | 749 | N.D |
| Deoxyribonuclease II (Fragment) | 683 | 284 |
| Transmembrane protein | 443 | N.D |
| Beta-glucuronidase | 419 | N.D |
| RNA-binding protein 34 | 413 | N.D |
| Beta-galactosidase (Fragment) | 338 | N.D |
| Carboxypeptidase | 292 | N.D |
| Attractin | 289 | 251 |
| Di-N-acetylchitobiase | 267 | N.D |
| AGA | 265 | N.D |
| Alpha-galactosidase | 264 | N.D |

N.D.: Not determined

Overall, the yield was about 90% or more, the purity was about 99% or more, and the HCP removal capacity was about 1.0 LRV or more.

### Example 9: Secondary Ultrafiltration/Diafiltration (UF/DF)

The supernatant of the caprylate precipitate was adjusted to pH and conductivity suitable for secondary anion exchange chromatography through secondary ultrafiltration/diafiltration. After the second ultrafiltration/diafiltration, the concentration factor increased by about 2 times, the buffer exchange volume was 3 DV or more, the pH increased from 4.5 to about 7.5, and the conductivity decreased from 25 mS/cm to 6 mS/cm or less.

Specifically, pH 7.5, 20 mM histidine buffer was allowed to flow using Pellicon 3 Ultracel C screen (Cat No. P3C030C01) membrane from Merck having a cut-off value of 30 to 50 kDa, to achieve an equilibrium state, the supernatant of the caprylate precipitate was concentrated by 2 times compared to the initial volume, the buffer was exchanged with 2 DV or more of pH 7.5 20 mM histidine buffer, the process solution was recovered when the conductivity reached 6 mS/cm or less, and then the pH of the recovered process solution was adjusted to 7.5.

### Example 10: Secondary Anion Exchange Chromatography

In order to remove process-related impurities such as heparin, caprylate, solvent and detergent from the second ultrafiltration/diafiltration solution, secondary anion exchange chromatography was performed using, as strong anion exchange resins, Fractogel EMD TMAE (M) and Fractogel EMD TMAE (S).

Specifically, the resin was subjected to CIP with 5 CV of 0.5 N NaOH, 15 CV of 20 mM histidine equilibration buffer (pH 7.5±0.5, 50 mM NaCl) was allowed to flow to achieve an equilibration state, and the secondary UF/DF solution was loaded. Then, 10 CV of 20 mM histidine equilibration buffer (pH 7.0±0.5, 50 mM NaCl) was injected to achieve an equilibrium state and 7 CV of 20 mM histidine elution buffer (pH 7.0±0.5, 150±10 mM NaCl) was injected.

Then, 5 CV of 20 mM histidine column wash buffer (CW buffer, pH 7.0±0.5, 2000±200 mM NaCl) was injected, CIP was performed with 5 CV of 0.5 N NaOH, and an equilibration buffer was allowed to flow at 10 CV to achieve an equilibrium state.

Overall, the yield was about 90% or more, the purity was about 99% or more, and there was almost no difference between the resins.

In addition, the result of test performed while fixing the concentration of NaCl in the eluate at 150 mM and changing the pH from 6.8 to 7.7 showed that heparan-N-sulfatase could be efficiently purified in all cases, as shown in Table 7.

TABLE 7
Purification effect of AEX depending on pH

| | | Protein content (UV) | | | | |
|---|---|---|---|---|---|---|
| | Loading | Loading sample | | Elution pool | | |
| Exp. | solution | Vol. | Conc. | Vol. | Conc. | Yield |
| No. | pH | (mL) | (mg/mL) | (mL) | (mg/mL) | (%) |
| 1 | 7.5 | 117.8 | 1.10 | 78.5 | 1.53 | 92.73 |
| 2 | 7.3 | 117.8 | 1.15 | 78.5 | 1.56 | 90.43 |
| 3 | 7.7 | 117.8 | 1.12 | 78.5 | 1.55 | 92.26 |
| 4 | 7.0 | 25 | 1.73 | 39.3 | 1.05 | 95.29 |
| 5 | 6.8 | 22 | 1.98 | 39.3 | 1.04 | 93.71 |
| 6 | 7.2 | 26 | 1.61 | 39.3 | 1.03 | 96.58 |

In addition, the result of test performed while fixing the pH at 7.5 and changing the concentration of NaCl in the eluate to 130 to 150 mM showed that heparan-N-sulfatase could be efficiently purified in all cases, as shown in Table 8.

TABLE 8
Purification effect of AEX depending on
the concentration of NaCl in the eluate

| | Protein content (UV) | | | | | Purity |
|---|---|---|---|---|---|---|
| | Loading | | | | | Elution |
| NaCl | sample | | Elution pool | | | pool |
| concentration | Vol. | Conc. | Vol. | Conc. | Yield | Purity |
| (mM) | (mL) | (mg/mL) | (mL) | (mg/mL) | (%) | (%) |
| 1 | 142.7 | 0.55 | 80 | 1.03 | 106.0 | 99.19 |
| 2 | 142.7 | 0.55 | 80 | 0.93 | 95.7 | 99.06 |
| 3 | 142.7 | 0.55 | 160 | 0.51 | 104.9 | 99.96 |

### Example 11: Nanofiltration

Finally, in order to remove viruses by filtration, nanofiltration was performed using nanofilters from Merck KGaA, Asahi, and Sartorius. Although there was no difference between the products from the manufacturers, the recovery rate of the nanofilter from Sartorius was rather high and the overall yield was more than 90%.

### Example 12: Tertiary Ultrafiltration/Diafiltration (UF/DF)

Tertiary ultrafiltration/diafiltration was performed for final formulation to achieve high concentration and buffer exchange.

Specifically, 4.2 mM histidine buffer (pH 8.2, 93.75 mM NaCl) was allowed to flow using Pellicon 3 Ultracel C screen (Cat. No. P3C030C01) membrane from Merck KGaA having a cut-off value of 30 to 50 kDa, to achieve an equilibrium state, and the secondary AEX eluate was concentrated to about 5 mg/mL and then recovered.

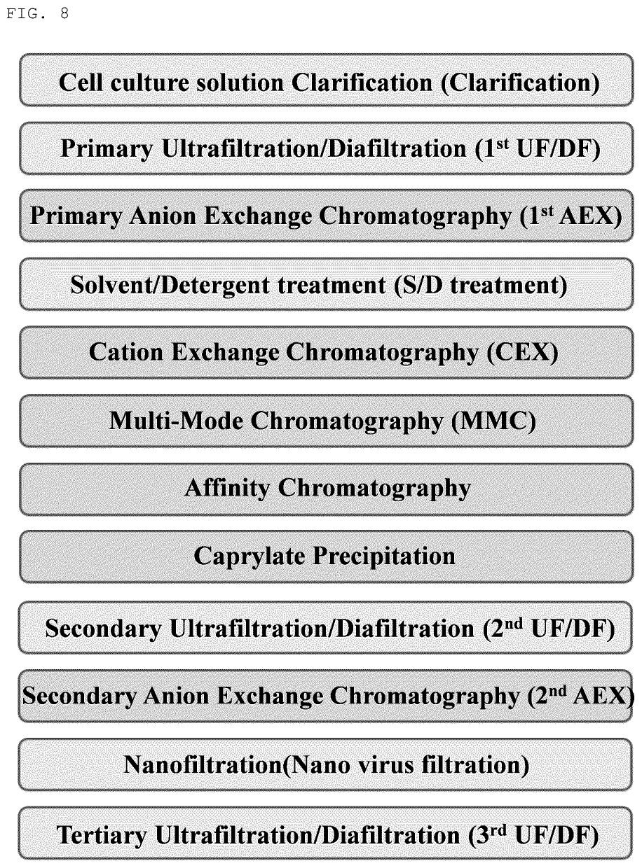

An overall process diagram according to a specific embodiment of the present invention is shown in FIG. 8.

Although specific configurations of the present invention have been described in detail, those skilled in the art will appreciate that the present invention can be implemented in modified forms without departing from the essential characteristics of the present invention. Therefore, the embodiments described above should be considered from an illustrative rather than a limiting point of view. The scope of the present invention is defined in the claims rather than the foregoing description and all differences equivalent thereto would be construed as falling within the scope of the present invention.

### INDUSTRIAL APPLICABILITY

The method for purifying heparan-N-sulfatase according to the present invention can greatly improve the purity, safety and stability of the produced heparan-N-sulfatase by efficiently removing HCP and other impurities, thus being highly suitable for efficient production of heparan-N-sulfatase for use in enzyme replacement therapy.

1. A method for purifying heparan-N-sulfatase from a heparan-N-sulfatase-containing solution including at least one impurity, the method comprising:
performing multi-mode chromatography (MMC) to obtain an eluate; and
performing caprylate precipitation to obtain a supernatant.

2. The method according to claim 1, wherein the heparan-N-sulfatase-containing solution is a cell culture solution.

3. The method according to claim 1, wherein the multi-mode chromatography is implemented by simultaneously performing cation exchange chromatography (CEX) and hydrophobic interaction chromatography (HIC).

4. The method according to claim 3, wherein a resin used for the multi-mode chromatography (MMC) is a Capto MMC resin or a Capto adhere resin.

5. The method according to claim 3, wherein the multi-mode chromatography (MMC) comprises two or more washing steps after the heparan-N-sulfatase binds to the resin.

6. The method according to claim 5, wherein, in two washing steps, a pH of buffer used in a subsequent washing step is higher than a pH of buffer used in a primary washing step.

7. The method according to claim 1, wherein the caprylate precipitation is performed by adding caprylate at a concentration of 1 to 20 mM.

8. The method according to claim 1, further comprising:
performing affinity chromatography to obtain an eluate,
between performing multi-mode chromatography (MMC) to obtain an eluate and performing caprylate precipitation to obtain a supernatant.

9. The method according to claim 8, further comprising:
performing primary anion exchange chromatography (AEX) to obtain an eluate;
solvent/detergent treatment; and
performing cation exchange chromatography (CEX) to obtain an eluate,
prior to performing multi-mode chromatography to obtain an eluate.

10. The method according to claim 9, wherein a resin used for the primary anion exchange chromatography (AEX) is a weak anion exchange resin or a strong anion exchange resin.

11. The method according to claim 9, further comprising:
performing secondary anion exchange chromatography (AEX) to obtain an eluate,
after performing caprylate precipitation to obtain a supernatant.

12. The method according to claim 11, further comprising nanofiltration,
after performing secondary anion exchange chromatography (AEX) to obtain an eluate.

13. The method according to claim 12, further comprising ultrafiltration/diafiltration (UF/DF),
at least one time selected from the steps consisting of before performing primary anion exchange chromatography to obtain an eluate; between performing caprylate precipitation to obtain a supernatant and performing secondary anion exchange chromatography (AEX) to obtain an eluate; and after nanofiltration.


SEQUENCE LISTING (1 sequence)

SEQ ID NO: 1 - protein, 502 aa, Homo sapiens
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL